(19) United States
(12) Patent Application Publication
(10) Pub. No.: US 2018/0282417 A1
(43) Pub. Date: Oct. 4, 2018

(54) TUMOR BURDEN AS MEASURED BY CELL FREE DNA

(71) Applicant: MedImmune, LLC, Gaithersburg, MD (US)

(72) Inventors: Brandon Higgs, Gaithersburg, MD (US); Koustubh Ranade, Gaithersburg, MD (US); Carlos Bais, Gaithersburg, MD (US); Philip Brohawn, Gaithersburg, MD (US); Michael Kuziora, Gaithersburg, MD (US); Rajiv Raja, Gaithersburg, MD (US)

(21) Appl. No.: 15/941,358

(22) Filed: Mar. 30, 2018

Related U.S. Application Data
(60) Provisional application No. 62/479,896, filed on Mar. 31, 2017.

(51) Int. Cl. C07K 16/28; C12Q 1/6886

(52) U.S. Cl. CPC C07K 16/2827; A61K 2039/545; C12Q 1/6886

(57) ABSTRACT

Disclosed are methods for treating cancer (e.g., solid tumor cancers, lung cancer, bladder head and neck cancer) with an anti-PD-L1 antibody in a patient identified as being responsive to anti-PD-L1 antibody therapy by detecting a mutation in one or more disclosed circulating tumor DNA (ctDNA) markers. Also disclosed are methods for determining the efficacy of anti-PD-L1 therapeutic antibody treatment in a patient having lung cancer or bladder cancer comprising detecting variant allele frequency in ctDNA in plasma samples and determining the difference of the variant allele frequency in ctDNA between the first and at least second plasma samples, wherein a decrease in the variant allele frequency in the at least second plasma sample relative to the first plasma sample identifies the anti-PD-L1 antibody treatment as effective. The disclosure also provides methods of identifying a subject having a cancer responsive to a therapy comprising an anti-PD-L1 antibody by detecting the expression of a mutation in one or more circulating tumor DNA (ctDNA) markers.

### BACKGROUND OF THE INVENTION

Cancer remains a major cause of death despite consistent therapeutic advances such as immunooncology therapies. Evaluation of patient response to therapeutic intervention can be slow and is typically determined by measuring change in tumor size several months after initiation of therapy. Application of next generation sequencing (NGS) technology in the diagnosis, prognosis, and treatment of cancer allows for a more rapid and patient-specific evaluation of disease status and therapeutic options. Under normal conditions, cell-free DNAs (cfDNA) are observable in the blood only in low amounts, however they are not efficiently cleared in response to exhaustive exercise, inflammation, or occurrence of disease. For example, circulating DNAs deriving from cancer cells represents a distinct and measurable component of the cfDNA in cancer patients. This circulating tumor DNA (ctDNA) fraction of cfDNA can be useful for classifying tumors and cancer disease, such as stratifying cancer patients, allowing for administration of therapies that are more likely to be effective, as well as for modification of current therapies that are less likely to provide clinical improvement. As discussed below, analysis of ctDNA in subjects having cancer provides methods of diagnosis, prognosis, and treatment (e.g., predicting the responsiveness, determining a course of therapy) of cancer that improve upon existing technology.

### SUMMARY OF THE INVENTION

The disclosure relates to methods for treating cancer (e.g., lung cancer such as non-small cell lung cancer, bladder cancer, solid tumors, and the like) with an anti-PD-L1 antibody in a patient identified as having a cancer that expresses a mutation in one or more circulating tumor DNA (ctDNA) markers disclosed herein. The disclosure also provides methods for determining responsiveness of a cancer to therapeutic treatment that comprises an anti-PD-L1 antibody. Also provided are methods of identifying a cancer patient as a candidate for therapy comprising an anti-PD-L1 antibody.

In one aspect, the disclosure generally provides a method of treatment comprising administering an anti-PD-L1 antibody, or an antigen binding fragment thereof, to a patient identified as having a cancer that expresses a mutation in one or more circulating tumor DNA (ctDNA) markers comprising BRCA1, BRCA2, PIK3CA, NFE2L2, NOTCH1, ARID1A, APC, SMAD4, or KRAS. In some embodiments, the anti-PD-L1 antibody is durvalumab.

In one aspect, the disclosure generally provides a method of treatment comprising administering an anti-PD-L1 antibody, or an antigen binding fragment thereof, to a patient identified as having a lung cancer (e.g., non-small cell lung cancer) that expresses a mutation in one or more circulating tumor DNA (ctDNA) markers comprising BRCA1, BRCA2, NFE2L2, PIK3CA, ARID1A, APC, or NOTCH1. In one embodiment, the anti-PD-L1 antibody is durvalumab.

In one aspect, the disclosure generally provides a method of treatment comprising administering an anti-PD-L1 antibody, or an antigen binding fragment thereof, to a patient identified as having a bladder cancer that expresses a mutation in one or more circulating tumor DNA (ctDNA) markers comprising BRCA1, BRCA2, ARID1A, APC, PIK3CA, and NOTCH1. In some embodiments, the anti-PD-L1 antibody is durvalumab.

In another aspect, the disclosure generally provides a method of treatment comprising administering an anti-PD-L1 antibody, or an antigen binding fragment thereof, to a patient identified as having a head and neck cancer tumor that expresses one or more markers disclosed herein. In some embodiments, the head and neck cancer expresses a mutation in one or more circulating tumor DNA (ctDNA) markers comprising NFE2L2, APC, and PIK3CA. In one embodiment, the anti-PD-L1 antibody is durvalumab.

In one aspect, the disclosure generally provides a method of treatment comprising administering an anti-PD-L1 antibody, or an antigen binding fragment thereof, to a patient identified as having a solid tumor cancer (e.g., sarcoma, carcinoma, and lymphoma) that expresses one or more markers disclosed herein. In one embodiment, the anti-PD-L1 antibody is durvalumab.

In another aspect, the disclosure provides a method of treatment comprising administering durvalumab or an antigen binding fragment thereof to a patient identified as having a non-small cell lung cancer tumor that expresses one or more markers that comprise BRCA1, BRCA2, NFE2L2, PIK3CA, ARID1A, APC, or NOTCH1.

In another aspect, the invention provides a method of treatment comprising administering durvalumab or an antigen binding fragment thereof to a patient identified as having a bladder cancer tumor that expresses one or more markers that comprises BRCA1, BRCA2, ARID1A, APC, PIK3CA, or NOTCH1.

In another aspect, the invention provides a method of treatment comprising administering durvalumab or an antigen binding fragment thereof to a patient identified as having a head and neck cancer tumor that expresses one or more of NFE2L2, APC, and PIK3CA.

In a further aspect, the invention provides a method of identifying a subject having a cancer responsive to an anti-PD-L1 therapy, the method comprising detecting the expression of a mutation in one or more circulating tumor DNA (ctDNA) markers comprising BRCA1, BRCA2, PIK3CA, NFE2L2, NOTCH1, SMAD4, ARID1A, APC, or KRAS in a sample obtained from the subject.

In another aspect, the disclosure herein provides a method of treating a patient identified patient having cancer, the method comprising: detecting variant allele frequency in one or more ctDNA markers in a first plasma sample taken from the patient at a first time point, administering an anti-PD-L1 therapeutic antibody to the patient after obtaining the first plasma sample, detecting variant allele frequency in one or more ctDNA markers in at least a second plasma sample taken from the patient at least at a second time point after administration of the anti-PD-L1 therapeutic antibody, and determining the difference of the variant allele frequency in one or more ctDNA markers between the first and at least second plasma samples, wherein a decrease in the variant allele frequency in the at least second plasma sample relative to the first plasma sample identifies the anti-PD-L1 antibody treatment as effective, and wherein the one or more circulating tumor DNA (ctDNA) markers comprise BRCA1, BRCA2, PIK3CA, NFE2L2, NOTCH1, SMAD4, ARID1A, APC, or KRAS.

In some embodiments of this aspect, the method identifies a subject having a lung cancer responsive to an anti-PD-L1 therapy, comprising detecting the expression of a mutation in one or more circulating tumor DNA (ctDNA) markers comprising BRCA1, BRCA2, PIK3CA, NFE2L2, NOTCH1, SMAD4, ARID1A, APC, or KRAS in a sample obtained from the subject. In further embodiments, the method comprises detecting the expression of a mutation in one or more circulating tumor DNA (ctDNA) markers comprising BRCA1, BRCA2, NFE2L2, PIK3CA, ARID1A, APC, or NOTCH1. In yet further embodiments, the method comprises detecting the expression of a mutation in one or more circulating tumor DNA (ctDNA) markers comprising BRCA2 or NFE2L2.

In some embodiments of this aspect, the method identifies a subject having a bladder cancer responsive to an anti-PD-L1 therapy, comprising detecting the expression of a mutation in one or more circulating tumor DNA (ctDNA) markers comprising BRCA1, BRCA2, PIK3CA, NFE2L2, NOTCH1, SMAD4, ARID1A, APC, or KRAS in a sample obtained from the subject. In further embodiments, the method comprises detecting the expression of a mutation in one or more circulating tumor DNA (ctDNA) markers comprising one or more of BRCA1, BRCA2, ARID1A, APC, PIK3CA, or NOTCH1.

In some embodiments of this aspect, the method identifies a subject having head and neck cancer responsive to anti-PD-L1 therapy, comprising detecting the expression of a mutation in one or more circulating tumor DNA (ctDNA) markers of NFE2L2, APC, and PIK3CA.

In some of the above embodiments, the method further comprises detecting PD-L1 expression in the tumor.

In various embodiments of the above aspects or any other aspect of the disclosure described herein, the patient is identified as responsive to durvalumab. In some embodiments of any aspect of the disclosure herein, the patient is further identified as having a tumor expressing PD-L1.

In various embodiments of the above aspects, treatment may comprise administration of at least about 0.1, about 0.3, about 1, about 3, about 10, or about 15 mg/kg durvalumab, or an antigen-binding fragment thereof. In other embodiments, at least about 1 mg/kg, 3 mg/kg, 10 mg/kg, or 15 mg/kg durvalumab, or an antigen-binding fragment thereof, is administered. In other embodiments, the administration is repeated about every 14 or 21 days. In other embodiments, at least two, three, four, or five doses is administered.

In a further aspect, the disclosure provides a method for characterizing the responsiveness of a cancer in a subject to an anti-PD-L1 antibody treatment, the method comprising: detecting variant allele frequency in ctDNA in a first plasma sample taken from the subject at a first time point, detecting variant allele frequency in ctDNA in at least a second plasma sample taken from the subject at least at a second time point, and determining the difference of the variant allele frequency in ctDNA between the first and at least second plasma samples, wherein a decrease in the variant allele frequency in the at least second plasma sample relative to the first plasma sample characterizes the cancer as responsive to anti-PD-L1 antibody treatment.

In another aspect, the disclosure herein provides a method of treating a patient having a cancer comprising, identifying whether the patient will be responsive to an anti-PD-L1 antibody by detecting the expression of a mutation in one or more circulating tumor DNA (ctDNA) markers comprising BRCA1, BRCA2, PIK3CA, NFE2L2, NOTCH1, SMAD4, ARID1A, APC, or KRAS, and treating the patient with a therapy other than an anti-PD-L1 antibody if the one or more ctDNA markers is not expressed.

In some embodiments, the method further comprises administering the anti-PD-L1 antibody to the subject after the first plasma sample is taken from the subject. In some embodiments, the variant allele frequency in ctDNA is determined by total mutation count in the first sample and in at least the second sample. In some embodiments, the variant allele frequency in ctDNA is determined by the mean variant allele frequency in the first sample and in at least the second sample.

In various embodiments of the above aspects or any other aspect of the invention herein, ctDNA markers are detected using NGS techniques. In various embodiments of the above aspects or any other aspect of the invention herein, the ctDNAs are obtained from the blood of the cancer patient.

In various embodiments of the aspects described herein, the cancer comprises a lung cancer selected from the group consisting of non-small cell lung cancer, squamous cell carcinoma, non-squamous cell carcinoma, adenocarcinoma, large cell carcinoma, adenosquamous carcinoma or sarcomatoid carcinoma.

Other features, aspects, embodiments, and advantages provided by the disclosure will be apparent from the detailed description that follows.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “anti-PD-L1 antibody” is meant an antibody or antigen binding fragment thereof that selectively binds a PD-L1 polypeptide. Exemplary anti-PD-L1 antibodies are described for example at U.S. Pat. No. 8,779,108 and U.S. Pat. No. 9,493,565, which is herein incorporated by reference. Durvalumab is an exemplary PD-L1 antibody. Following successful treatment with durvalumab, a patient achieves disease control (DC). Disease control can be a complete response (CR), partial response (PR), or stable disease (SD).

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≥50% relative to baseline. Confirmation can be obtained using a consecutive repeat assessment at least 4 weeks from the date of first documentation.

“Stable disease” (SD) indicates a decrease in tumor burden of 50% relative to baseline cannot be established and a 25% increase compared to nadir cannot be established.

“Progressive disease” (PD) refers to an increase in tumor burden ≥25% relative to the minimum recorded (nadir). Confirmation can be obtained by a consecutive repeat assessment at least 4 weeks from the date of first documentation. New, non-measurable lesions do not define PD.

“ATLANTIC” means A Global Study to Assess the Effects of MEDI4736 (Durvalumab) in Patients With Locally Advanced or Metastatic Non Small Cell Lung Cancer (ClinicalTrials.gov Identifier: NCT0208742)

“CP1108” means A Phase 1/2 Study to Evaluate MEDI4736 (ClinicalTrials.gov Identifier: NCT01693562). As used herein, data from CP1108 include NSCLC and bladder cancer results.

By “PD-L1 polypeptide” is meant a polypeptide or fragment thereof having at least about 85%, 95% or 100% amino acid identity to NCBI Accession No. NP_001254635 and having PD-1 and CD80 binding activity.

| PD-L1 polypeptide sequence | |
|---|---|
| NCBI ACCESSION NO. NP_001254635 | |
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

By “PD-L1 nucleic acid molecule” is meant a polynucleotide encoding a PD-L1 polypeptide. An exemplary PD-L1 nucleic acid molecule sequence is provided at NCBI Accession No. NM_001267706.

| PD-L1 nucleic acid sequence | |
|---|---|
| NCBI ACCESSION NO. NM_001267706 mRNA | |
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize Digestion of antibodies with the enzyme, pepsin, results in the F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “sample” is meant a biological sample derived from any tissue, cell, fluid, or other material derived from an organism. In certain embodiments, a biological sample is a blood or plasma sample.

A “biomarker” or “marker” as used herein typically refers to a circulating tumor DNA (ctDNA) associated with a cancer. In embodiments, a ctDNA marker comprises a variant allele (mutation) of a gene that is associated with a cancer (e.g., an oncogene). In some embodiments a ctDNA marker is differentially present in a biological sample obtained from a subject having a disease (e.g., lung cancer) relative to the level present in a control sample or reference. In some embodiments a ctDNA marker is differentially present in a biological sample obtained from a subject prior to treatment of a disease (e.g., lung cancer) relative to the level present in a sample obtained from the same subject during or after treatment of a disease.

The methods disclosed herein comprise detection of ctDNA markers and may include detection of the total number of ctDNA counts for a particular marker or set of markers, detection of a change in the mean ctDNA marker frequency for a particular marker or set of markers, or detection of the presence of a particular ctDNA marker or set of ctDNA markers. Accordingly, in any of the aspects and embodiments disclosed herein, detection of the presence, number, or change in frequency of one or more of the ctDNA markers.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected, which in the various aspects and embodiments disclosed herein, comprises ctDNA. As discussed herein the detection of ctDNA markers (i.e., ctDNAs comprising variant/mutant alleles) can be used to assess variant allele frequency (VAF), change in the mean VAF, total mutation burden, and/or development of new driver mutations (i.e., mutation within a gene conferring a growth advantage) in a patient who may be selected for treatment, or who has undergone treatment, with an anti-PD-L1 antibody.

In the methods disclosed herein, the detection of ctDNA may be performed on samples that are derived from a patient once, or at a plurality of time points. For example, patient samples may be obtained prior to treatment (e.g., during screening and diagnosis), during treatment (e.g., prior to or following administration of a therapeutic dose), and/or following the course of treatment.

By “disease” is meant any condition or disorder that damages or interferes with the normal function of a cell, tissue, or organ. In the aspects and embodiments disclosed herein, the disease is typically a cancer such as, for example, a solid tumor cancer. In some embodiments, the cancer may include lung cancer, bladder cancer, and/or head and neck cancer. Lung cancer includes small cell lung cancer (SCLC) and non-small cell lung cancer (NSCLC). There are three main subtypes of NSCLC: squamous cell carcinoma, adenocarcinoma, and large cell (undifferentiated) carcinoma. Other subtypes include adenosquamous carcinoma and sarcomatoid carcinoma. In certain aspects, the NSCLC is unresectable, late stage (e.g., stage III) NSCLC. In some specific aspects, these patients have not progressed following definitive chemoradiation therapy. Head and neck cancer includes laryngeal and hypopharyngeal cancer, nasal cavity and paranasal sinus cancer, nasopharyngeal cancer, oral and oropharyngeal cancer, and salivary gland cancer. Bladder cancer includes urothelial carcinoma (also called transitional cell carcinoma), squamous cell carcinoma, adenocarcinoma, sarcoma, and small cell anaplastic cancer.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “reference” is meant a standard of comparison.

By “responsive” in the context of therapy is meant susceptible to treatment.

By “specifically binds” is meant a compound (e.g., antibody) that recognizes and binds a molecule (e.g., polypeptide), but which does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶M⁻¹, or more preferably higher than 10⁸M⁻¹. If necessary, non-specific binding can be reduced without substantially affecting specific binding by varying the binding conditions. The appropriate binding conditions such as concentration of antibodies, ionic strength of the solution, temperature, time allowed for binding, concentration of a blocking agent (e.g., serum albumin, milk casein), etc., may be optimized by a skilled artisan using routine techniques.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, ameliorating, or slowing the progression of a disorder or disease and/or symptoms associated with a disorder or disease. It will be appreciated that, although not precluded, treating a disorder, disease, or condition does not require that the disorder, disease, or condition or associated symptoms be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a summary of all ctDNA variants detected in plasma samples of non-small cell lung cancer (NSCLC) patients at baseline prior to treatment with anti-PD-L1 antibody (durvalumab).

FIG. 2 plots mean variant allele frequency of ctDNA at the time of initial patient screening. Responders show a decrease in mean VAF following anti-PD-L1 antibody therapy (“Postdose”), patients having stable disease generally exhibit a smaller decrease in mean VAF than responsive patients, while progressive disease patients generally show do not show a decrease in mean VAF following anti-PD-L1 antibody therapy. Each line represents a patient in the respective response groups (PR; PD; or SD).

FIG. 3A demonstrates that a decrease in mean VAF following therapy correlates with increased chance of overall survival (OS) in NSCLC patients. In the bar graph, the numbers above the bars is the time (weeks) when the evaluation of PR is made. FIG. 3B shows that NSCLC patients having a decrease in mean VAF generally have a greater chance of longer progression free survival and overall survival relative to patients exhibiting an increase in mean VAF.

FIG. 4 identifies that NSCLC patients from study 1108 responsive (PR) to treatment have a decrease in tumor burden as demonstrated by total ctDNA mutation counts, while patients with stable or progressive disease (PD/SD) typically show an increase in total ctDNA mutation counts.

FIG. 5 identifies the type and number of counts of new ctDNA mutations detected in PR, SD, and PD NSCLC patents following anti-PD-L1 antibody therapy.

FIG. 6 illustrates exemplary new driver mutations appearing in two non-responsive NSCLC patients (PD) following anti-PD-L1 antibody therapy.

FIG. 7 depicts the response rate to anti-PD-L1 antibody therapy of patients having ctDNA variants BRCA2, PIK3CA, NFE2L2, NOTCH1, SMAD4, and KRAS identified in baseline screening of NSCLC patients following anti-PD-L1 antibody therapy.

FIG. 8 provides a summary of all ctDNA variants detected in plasma samples of bladder cancer patients at baseline prior to treatment with anti-PD-L1 antibody (durvalumab). The five most common genes containing non-synonymous variants or copy number amplifications are TP53, ARID1A, PIK3CA, ERBB2, and TERT.

FIG. 9 depicts the response rate to anti-PD-L1 antibody therapy of patients having ctDNA variants BRCA2, PIK3CA, NOTCH1, SMAD4, and KRAS identified in baseline screening of bladder cancer patients.

FIG. 10 is a plot that identifies mean variant allele frequency of ctDNA at the time of initial bladder cancer patient screening. Responders show a decrease in mean VAF following anti-PD-L1 antibody therapy (“Dose 4”), patients having stable disease generally exhibit a smaller decrease in mean VAF than responsive patients, while progressive disease patients generally show do not show a decrease in mean VAF following anti-PD-L1 antibody therapy (“Dose 4”). Each line represents a patient in the respective response groups (CR/PR; PD; or SD).

FIG. 11 shows that bladder cancer patients having a decrease in mean VAF generally have a greater chance of longer progression free survival and overall survival.

FIG. 12 demonstrates that a decrease in mean VAF following therapy correlates with responsive and stable disease in bladder cancer patients and an increase in overall survival (OS). In the bar graph, the numbers above the bars is the time (weeks) when the evaluation of PR is made.

FIG. 13 provides a summary of all ctDNA variants detected in plasma samples of NSCLC cancer patients at baseline clinical evaluation of treatment with anti-PD-L1 antibody (durvalumab) (ATLANTIC clinical trial). The most common variants are TP53, KRAS, EGFR, ARID1A, and PIK3CA (88 total samples).

FIG. 14 plots mean variant allele frequency of ctDNA at the time of initial patient screening. Data generated from both ATLANTIC and CP1108. Responders show a decrease in mean VAF following anti-PD-L1 antibody therapy (“Postdose”), patients having stable disease generally exhibit a smaller decrease in mean VAF than responsive patients, while progressive disease patients generally show do not show a decrease in mean VAF following anti-PD-L1 antibody therapy. Each line represents a patient in the respective response groups (CR/PR; PD; or SD).

FIG. 15 demonstrates that a decrease in mean VAF following therapy correlates with increased chance of overall survival (OS) in NSCLC patients. Data generated from both ATLANTIC and CP1108.

FIG. 16 shows that ATLANTIC patients having a decrease in mean VAF generally have a greater chance of longer progression free survival and overall survival relative to patients exhibiting an increase in mean VAF.

FIG. 17 provides individual patient response data for NSCLC and UBC patients in months

FIG. 18 depicts the response rate to anti-PD-L1 antibody therapy of patients having ctDNA variants NFE2L2, RET, EGFR, MET, and PIK3CA, in NSCLC patients following anti-PD-L1 antibody therapy. Data generated from both ATLANTIC and CP1108.

FIG. 19 depicts the response rate to anti-PD-L1 antibody therapy from pooled patient data having ctDNA variants NFE2L2, BRCA2, RET, PIK3CA, NOTCH1, EGFR, and KRAS, in NSCLC patients following anti-PD-L1 antibody therapy.

### SEQUENCES

Durvalumab light chain variable region amino acid sequence: SEQ ID NO: 1

Durvalumab heavy chain variable region amino acid sequence: SEQ ID NO: 2.

Durvalumab heavy chain variable region amino acid sequence of CDR1, CDR2, and CDR3: SEQ ID NOs: 3-5.

Durvalumab light chain variable region amino acid sequence of CDR1, CDR2, and CDR3: SEQ ID NOs: 6-8.

### DETAILED DESCRIPTION OF THE INVENTION

The invention is based, at least in part, on the discovery that mutations (variant allele frequency, or “VAF”) in circulating tumor DNA (ctDNA) are detectable in samples obtained from a subject suffering from a cancer (e.g., lung cancer, bladder cancer, or head and neck cancer) and that VAF in ctDNAs can be used to identify patients who are likely to respond to treatment with an anti-PD-L1 antibody. The methods disclosed herein leverage the technological advances provided by next generation sequencing and correlate specific mutations in ctDNA and/or the observed mutational burden in ctDNA that is isolated from plasma samples to the responsiveness of the cancer to the anti-PD-L1 antibody therapy. The methods disclosed herein utilize ctDNA which allows for a relatively non-invasive method that can rapidly assess tumor DNA alterations and the likelihood of positive clinical response to therapy (e.g., comparing mutational burden in ctDNA before, during, and/or after therapy). Methods utilizing ctDNAs provide a representative illustration of all tumor lesions in a patient as well as tumor heterogeneity as such methods do not rely on biopsies or immunohistochemical techniques applied to individual tissue and/or tumor samples. The methods disclosed herein provide for repeat sampling, which allows for more efficient and rapid monitoring of the therapeutic response to treatment, mutational status of the cancer, and for development of molecular resistance prior to any typical clinical manifestations associated with disease relapse.

Thus, in various aspects, the disclosed methods comprising detection of ctDNA markers provide for the measurement of the mutational burden, allow for the identification of specific ctDNA mutations that can be predictive of positive clinical response prior to or during therapy, and also allow for modification of therapy when no signs of responsiveness to a current therapy are observable.

In one aspect, the disclosure provides methods for treating a cancer with an anti-PD-L1 antibody in a patient identified by detecting a ctDNA marker comprising a mutation in one or more of BRCA1, BRCA2, PIK3CA, NFE2L2, NOTCH1, SMAD4, ARID1A, APC, and/or KRAS.

In some embodiments of this aspect, the disclosure provides methods for treating lung cancer (e.g., NSCLC) with an anti-PD-L1 antibody in a patient identified by detecting a ctDNA marker comprising a mutation in one or of BRCA1, BRCA2, PIK3CA, NFE2L2, NOTCH1, SMAD4, ARID1A, APC, and/or KRAS. In further embodiments, the methods for treating lung cancer (e.g., NSCLC) in a patient comprise identifying the patient by detecting a ctDNA marker comprising a mutation in one or more of BRCA2, NFE2L2, and NOTCH1.

In some embodiments of this aspect, the disclosure provides methods for treating bladder cancer with an anti-PD-L1 antibody in a patient identified by detecting a ctDNA marker comprising a mutation in one or more of BRCA1, BRCA2, PIK3CA, NFE2L2, NOTCH1, SMAD4, ARID1A, APC, and/or KRAS. In further embodiments, the methods for treating bladder cancer in a patient comprise identifying the patient by detecting a ctDNA marker comprising a mutation in BRCA1, BRCA2, ARID1A, APC, PIK3CA, or NOTCH1.

In some embodiments, the disclosure provides methods for treating head and neck cancer with an anti-PD-L1 antibody in a patient identified by detecting a ctDNA marker comprising a mutation in one or more of BRCA1, BRCA2, PIK3CA, NFE2L2, NOTCH1, SMAD4, and/or KRAS). In some embodiments, the ctDNA marker comprises NFE2L2, APC, or PIK3CA.

In the above aspects and embodiments, the methods may be performed in combination with detection of a PD-L1 biomarker.

The methods disclosed above comprise identifying patients having a cancer that is responsive to treatment with an anti-PD-L1 antibody. As such, the disclosure provides for aspects and embodiments that include methods of identifying a subject having a cancer that is responsive to an anti-PD-L1 antibody, where the methods comprise detecting the expression of a mutation in one or more circulating tumor DNA (ctDNA) markers as described herein and in the above aspects and embodiments.

### Characterizing Responsiveness to Anti-PD-L1 Antibody Therapy

In a further aspect, the disclosure provides a method for characterizing the responsiveness of a cancer in a subject, such as a lung cancer or bladder cancer, to anti-PD-L1 antibody treatment, wherein the method comprises detecting the variant allele frequency in ctDNA in a first plasma sample taken from the subject at a first time point, detecting the variant allele frequency in ctDNA in at least a second plasma sample comprising ctDNA taken from the subject at least at a second time point, and determining the difference of the variant allele frequency in ctDNA between the first and at least second plasma samples.

In some embodiments of this aspect, the variant allele frequency in ctDNA can be determined by total mutation count in the first and second samples. In some embodiments, the variant allele frequency in ctDNA can be determined using the mean variant allele frequency in the first and second samples.

The methods for characterizing responsiveness are based on differences in the total mutational burden in the ctDNA markers that are detected in the first and second samples. In some embodiments, the ctDNA markers may comprise difference in a mutation in one or more of BRCA1, BRCA2, PIK3CA, NFE2L2, NOTCH1, SMAD4, ARID1A, APC, and/or KRAS. In further embodiments the method may comprise detection of differences in the variant allele frequency, optionally in combination with detection of PD-L1, measured in one or more types of biological samples (e.g., tumor sample).

In embodiments, the method identifies a subject who is responsive to anti-PD-L1 antibody treatment by determining a decrease in the variant allele frequency in the at least second sample when compared to the first sample. In some embodiments, the method identifies a subject as responsive upon detection of a lower number of total mutation counts in the ctDNAs in the at least second sample relative to the total mutation counts in the ctDNAs in the first sample. In some embodiments, the method identifies a subject as responsive upon detection of a lower mean variant allele frequency in the ctDNAs in the at least second sample relative to the mean variant allele frequency in the ctDNAs in the first sample.

As exemplified herein ctDNA markers including, for example, BRCA1, BRCA2, PIK3CA, NFE2L2, NOTCH1, SMAD4, ARID1A, APC, and/or KRAS can be detected using any next generation sequencing technology known in the art.

### Selection of a Treatment Method

Subjects suffering from a cancer such as, for example, lung cancer (e.g., NSCLC), bladder cancer, or head and neck cancer may be screened for mutations in ctDNA of one or more of BRCA1, BRCA2, PIK3CA, NFE2L2, NOTCH1, SMAD4, ARID1A, APC, and/or KRAS, and optionally PD-L1 polynucleotide or polypeptide expression, in the course of selecting a treatment method. In some embodiments, subjects suffering from NSCLC who have a mutation in one or more of BRCA1, BRCA2, NFE2L2, PIK3CA, and/or NOTCH1 ctDNA may be identified as likely responders to anti-PD-L1 treatment. In some embodiments, subjects suffering from bladder cancer who have a mutation in BRCA1, BRCA2, ARID1A, APC, PIK3CA, or NOTCH1 ctDNA may be identified as likely responders to anti-PD-L1 treatment.

### B7-H1/PD-L1

B7-H1, also known as PD-L1, is a type I transmembrane protein of approximately 53 kDa in size. In humans B7-H1 is expressed on a number of immune cell types including activated and anergic/exhausted T cells, on naïve and activated B cells, as well as on myeloid dendritic cells (DC), monocytes and mast cells. It is also expressed on non-immune cells including islets of the pancreas, Kupffer cells of the liver, vascular endothelium and selected epithelia, for example airway epithelia and renal tubule epithelia, where its expression is enhanced during inflammatory episodes. B7-H1 expression is also found at increased levels on a number of tumors including, but not limited to breast, colon, colorectal, lung, renal, including renal cell carcinoma, gastric, bladder, non-small cell lung cancer (NSCLC), hepatocellular cancer (HCC), and pancreatic cancer, as well as melanoma.

B7-H1 is known to bind two alternative ligands, the first of these, PD-1, is a 50-55 kDa type I transmembrane receptor that was originally identified in a T cell line undergoing activation-induced apoptosis. PD-1 is expressed on activated T cells, B cells, and monocytes, as well as other cells of the immune system and binds both B7-H1 (PD-L1) and the related B7-DC (PD-L2). The second is the B7 family member B7-1, which is expressed on activated T cells, B cells, monocytes and antigen presenting cells.

Signaling via the PD-1/B7-H1 axis is believed to serve important, non-redundant functions within the immune system, by negatively regulating T cell responses. B7-H1 expression on tumor cells is believed to aid tumors in evading detection and elimination by the immune system. B7-H1 functions in this respect via several alternative mechanisms including driving exhaustion and anergy of tumor infiltrating T lymphocytes, stimulating secretion of immune repressive cytokines into the tumor micro-environment, stimulating repressive regulatory T cell function and protecting B7-H1 expressing tumor cells from lysis by tumor cell specific cytotoxic T cells.

### Anti-PD-L1 Antibodies

Antibodies that specifically bind and inhibit PD-L1 activity (e.g., binding to PD-1 and/or CD80) are useful for the treatment of lung cancer (e.g., non-small cell lung cancer

Durvalumab is an exemplary anti-PD-L1 antibody that is selective for B7-H1 and blocks the binding of B7-H1 to the PD-1 and CD80 receptors. Durvalumab can relieve B7-H1-mediated suppression of human T-cell activation in vitro and inhibits tumor growth in a xenograft model via a T-cell dependent mechanism. Other agents that could be used include agents that inhibit PD-L1 and/or PD-1 (AB or other).

Information regarding durvalumab (or fragments thereof) for use in the methods provided herein can be found in International Application Publication No. WO 2011/066389 A1, the disclosure of which is incorporated herein by reference in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC).

Durvalumab and antigen-binding fragments thereof for use in the methods provided herein comprises a heavy chain and a light chain or a heavy chain variable region and a light chain variable region. In a specific aspect, durvalumab or an antigen-binding fragment thereof for use in the methods provided herein comprises a light chain variable region comprising the amino acid sequence of SEQ ID NO:1 and a heavy chain variable region comprising the amino acid sequence of SEQ ID NO: 2. In a specific aspect, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined, Abm-defined or other CDR definitions known to those of ordinary skill in the art. In a specific aspect, durvalumab or an antigen-binding fragment thereof for use in the methods provided herein comprises the variable heavy chain and variable light chain CDR sequences of the 2.14H9OPT antibody as disclosed in U.S. Pat. No. 8,779,108; U.S. Pat. No. 9,493,565 ; and in WO 2011/066389 A1, which are herein incorporated by reference in their entirety.

Treatment with an Anti-PD-L1 Antibody

Patients identified as likely responders to anti-PD-L1 antibody therapy are administered an anti-PD-L1 antibody, such as durvalumab, or an antigen-binding fragment thereof. Durvalumab or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

In some embodiments, at least two doses of durvalumab or an antigen-binding fragment thereof are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, durvalumab or an antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In some embodiments, durvalumab or an antigen-binding fragment thereof is administered over a three-week treatment period, a six-week treatment period, over a nine-week treatment period, over a twelve-week treatment period, over a twenty-four-week treatment period, or over a one-year or more treatment period. In some embodiments, durvalumab or an antigen-binding fragment thereof is administered over a two-month treatment period, over a four-month treatment period, or over a six-month or more treatment period (e.g., during a maintenance phase).

The amount of durvalumab or an antigen-binding fragment thereof to be administered to the patient will depend on various parameters, such as the patient's age, weight, clinical assessment, tumor burden and/or other factors, including the judgment of the attending physician.

In certain aspects the patient is administered one or more doses of durvalumab or an antigen-binding fragment thereof wherein the dose is about 0.1 mg/kg. In certain aspects the patient is administered one or more doses of durvalumab or an antigen-binding fragment thereof wherein the dose is about 0.3 mg/kg. In certain aspects the patient is administered one or more doses of durvalumab or an antigen-binding fragment thereof wherein the dose is about 1 mg/kg. In certain aspects the patient is administered one or more doses of durvalumab or an antigen-binding fragment thereof wherein the dose is about 3 mg/kg. In certain aspects the patient is administered one or more doses of durvalumab or an antigen-binding fragment thereof wherein the dose is about 10 mg/kg. In certain aspects the patient is administered one or more doses of durvalumab or an antigen-binding fragment thereof wherein the dose is about 15 mg/kg.

In certain aspects the patient is administered at least two doses of durvalumab or an antigen-binding fragment thereof wherein the dose is about 0.1 mg/kg. In certain aspects the patient is administered at least two doses of durvalumab or an antigen-binding fragment thereof wherein the dose is about 0.3 mg/kg. In certain aspects the patient is administered at least two doses of durvalumab or an antigen-binding fragment thereof wherein the dose is about 1 mg/kg. In certain aspects the patient is administered at least two doses of durvalumab or an antigen-binding binding fragment thereof wherein the dose is about 3 mg/kg. In certain aspects the patient is administered at least two doses of durvalumab or an antigen-binding fragment thereof wherein the dose is about 10 mg/kg. In certain aspects the patient is administered at least two doses of durvalumab or an antigen-binding fragment thereof wherein the dose is about 15 mg/kg. In some embodiments, the at least two doses are administered about two weeks apart. In some embodiments, the at least two doses are administered about three weeks apart.

In certain aspects the patient is administered at least three doses of durvalumab or an antigen-binding fragment thereof wherein the dose is about 0.1 mg/kg. In certain aspects the patient is administered at least three doses of durvalumab or an antigen-binding fragment thereof wherein the dose is about 0.3 mg/kg. In certain aspects the patient is administered at least three doses of durvalumab or an antigen-binding fragment thereof wherein the dose is about 1 mg/kg. In certain aspects the patient is administered at least three doses of durvalumab or an antigen-binding fragment thereof wherein the dose is about 3 mg/kg. In certain aspects the patient is administered at least three doses of durvalumab or an antigen-binding fragment thereof wherein the dose is about 10 mg/kg. In certain aspects the patient is administered at least three doses of durvalumab or an antigen-binding fragment thereof wherein the dose is about 15 mg/kg. In some embodiments, the at least three doses are administered about two weeks apart. In some embodiment, the at least three doses are administered about three weeks apart.

In certain aspects, administration of durvalumab or an antigen-binding fragment thereof according to the methods provided herein is through parenteral administration. For example, durvalumab or an antigen-binding fragment thereof can be administered by intravenous infusion or by subcutaneous injection. In some embodiments, the administration is by intravenous infusion.

In certain aspects, durvalumab or an antigen-binding fragment thereof is administered according to the methods provided herein in combination or in conjunction with additional cancer therapies. Such therapies include, without limitation, chemotherapeutic agents such as Vemurafenib, Erlotinib, Afatinib, Cetuximab, Carboplatin, Bevacizumab, Erlotinib, or Pemetrexed, or other chemotherapeutic agents, as well radiation or any other anti-cancer treatments.

The methods provided herein can decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects provided herein, the administration of durvalumab can reduce a tumor size by at least 25%. In certain aspects provided herein, the administration of durvalumab can reduce a tumor size by at least 25% within about 6 weeks of the first treatment. In certain aspects provided herein, the administration of durvalumab can reduce a tumor size by at least 50%. In certain aspects provided herein, the administration of durvalumab can reduce a tumor size by at least 50% within about 10 weeks of the first treatment. In certain aspects provided herein, the administration of durvalumab can reduce a tumor size by at least 75%. In certain aspects provided herein, the administration of durvalumab can reduce a tumor size by at least 75% within about 10 weeks of the first treatment.

In certain aspects, use of the methods provided herein, i.e., administration of durvalumab or an antigen-binding fragment thereof can decrease tumor size within 6 weeks, within 7 weeks, within 8 weeks, within 9 weeks, within 10 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks of the first treatment.

In some embodiments, administration of 1 mg/kg of durvalumab or an antigen-binding fragment thereof (e.g., at least one dose, at least two doses, at least three doses, at least four doses, at least five doses, at least six doses, at least seven doses, at least eight doses, at least nine doses, at least ten doses, or more every two weeks or every three weeks) can be sufficient to reduce tumor size. However, as provided herein, larger doses can also be administered, for example, to optimize efficacy, number of doses necessary, or certain pharmacokinetic parameters.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population.

In certain aspects, a patient achieves disease control (DC). Disease control can be a complete response (CR), partial response (PR), or stable disease (SD).

In certain aspects, administration of durvalumab or an antigen-binding fragment thereof can increase progression-free survival (PFS).

In certain aspects, administration of durvalumab or an antigen-binding fragment thereof can increase overall survival (OS).

According to the methods provided herein, administration of durvalumab or an antigen-binding fragment thereof can result in desirable pharmacokinetic parameters. Total drug exposure can be estimated using the “area under the curve” (AUC). “AUC (tau)” refers to AUC until the end of the dosing period, whereas “AUC (inf)”refers to the AUC until infinite time. The administration can produce AUC (tau) of about 100 to about 2,500 d·μg/mL. The administration can produce a maximum observed concentration (Cmax) of about 15 to about 350 μg/mL. The half-life of the durvalumab or the antigen-binding fragment thereof can be about 5 to about 25 days. In addition, the clearance of the durvalumab or the antigen-binding fragment thereof can be about 1-10 ml/day/kg.

As provided herein, durvalumab or an antigen-binding fragment thereof can also decrease free B7-H1 levels. Free B7-H1 refers to B7-H1 that is not bound (e.g., by durvalumab). In some embodiments, B7-H1 levels are reduced by at least 80%. In some embodiments, B7-H1 levels are reduced by at least 90%. In some embodiments, B7-H1 levels are reduced by at least 95%. In some embodiments, B7-H1 levels are reduced by at least 99%. In some embodiments, B7-H1 levels are eliminated following administration of durvalumab or an antigen-binding fragment thereof. In some embodiments, administration of durvalumab or an antigen-binding fragment thereof reduces the rate of increase of B7-H1 levels as compared, e.g., to the rate of increase of B7-H1 levels prior to the administration of durvalumab or an antigen-binding fragment thereof.

In certain aspects, a patient having cancer can be treated with a therapy other than an anti-PD-L1 antibody, if the one or more ctDNA markers, as disclosed herein, is not expressed. These therapies can be, e.g., an immune checkpoint inhibitor, chemotherapy, radiotherapy, immune system agonists, DNA damage response (DDR) inhibitors, tyrosine kinase inhibitors, oncolytic viruses, cancer vaccines, adenosine production inhibitors, or antibody-drug conjugates.

The practice of the methods disclosed herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

The following examples provide an illustration of some of the aspects and embodiments described above, and are not intended to limit the scope of the claimed invention.

### EXAMPLES

### Materials and Method

Patient Samples.

Plasma samples were obtained from patients enrolled in a phase 1/2 clinical trial evaluating the safety, tolerability, and pharmacokinetics of anti-PD-L1 antibody (durvalumab) in subjects with advanced solid tumors. Plasma samples from 28 patients having lung cancer (non-small cell lung cancer), and from 29 patients having bladder cancer were obtained during patient screening prior to therapy (durvalumab at 10.0 mg/kg) and again at week 8, after the fourth therapeutic dose (administered at week 6).

ctDNA Assay/NGS.

Next generation sequencing and detection of ctDNA was performed using the Guardant360 gene panel (Guardant Health, Inc., Redwood City, Calif.). The panel includes 73 genes and provides mutant allele frequency for each detected SNV, indel, and fusion, as well as copy number for detected amplifications.

### Example 1

### Decrease in ctDNA Mean Variant Allele Frequency Observed in Responding NSCLC Patients

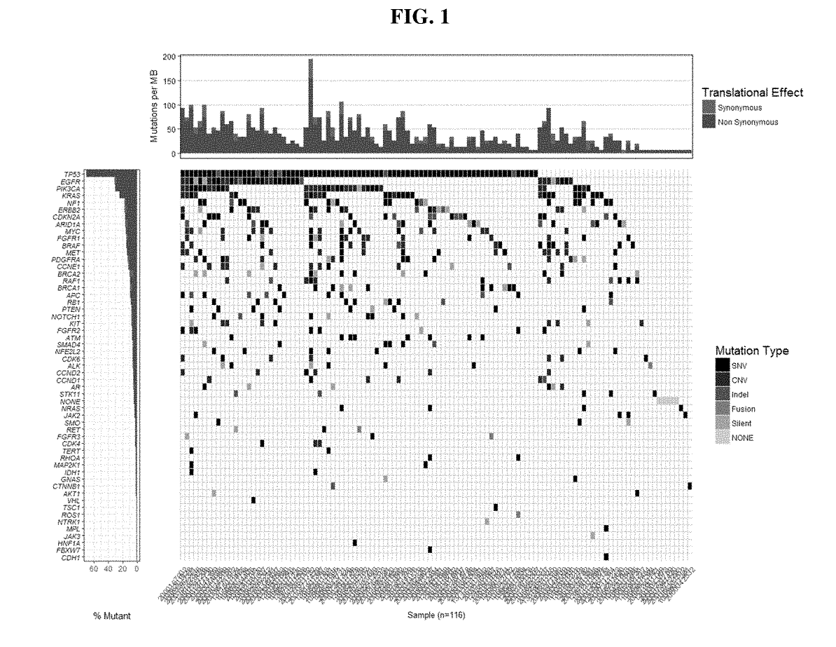

Analysis of patient samples confirms that ctDNA is detectable in cell-free DNA isolated from plasma. Analysis of samples obtained from 116 patients during screening identified variants in 96% of the samples (111/116), with several frequently observed variants, including TP53 (69% of samples), PIK3CA (29% of samples), EGFR (28% of samples), KRAS (24% of samples), and CDKN2A (16% of samples). See FIG. 1.

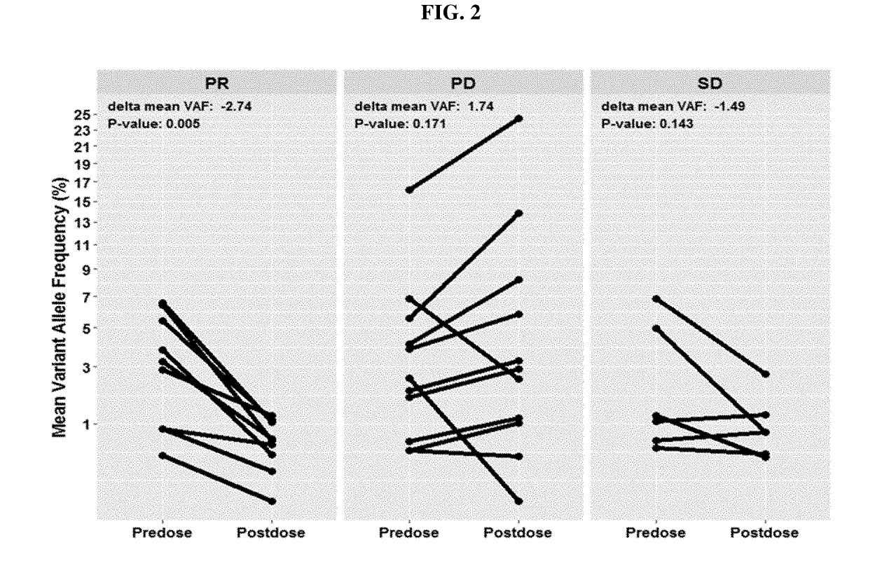

Changes in mean variant allele frequencies (VAF) during the course of therapy correlates with disease progression. Responding patients (PR) have a significant decrease (−1.6%, p=0.008) in ctDNA VAF after 4 doses/treatments while non-responders have an observable increase in mean VAF (+1.4%, p=0.05). See FIG. 2 which includes SNVs and indels with allele frequency ≥0.3% at screening and depicts the PFS probability and OS probability for patients exhibiting a mean decrease or increase in VAF after 6 weeks of durvalumab dosing. This data indicates that an observed reduction in mean VAF as early as 6 weeks after initiation of therapy may be predictive of longer PFS and OS with treatment comprising durvalumab.

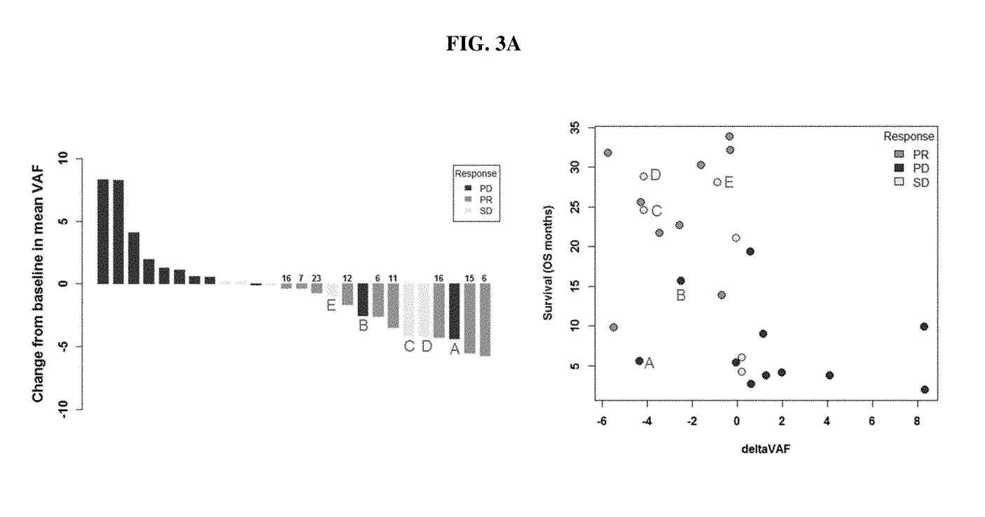

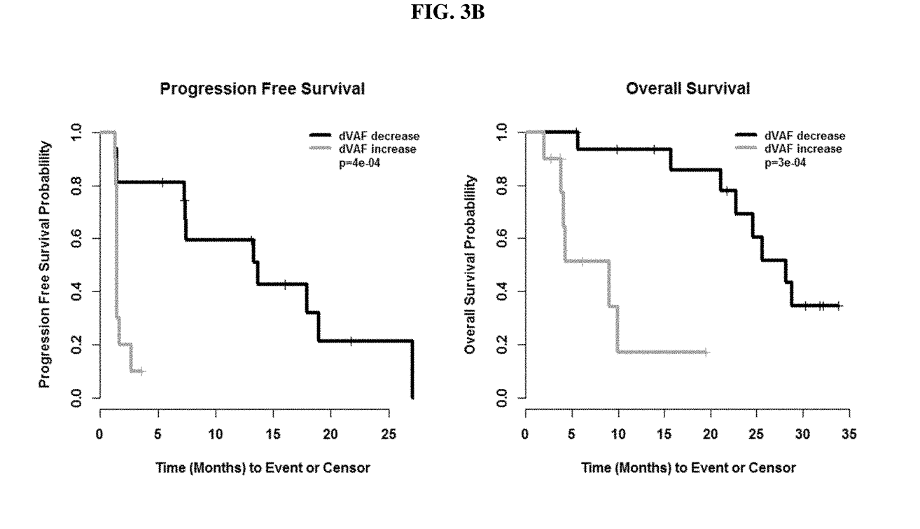

In FIG. 3A patient response is plotted as a function of the change in mean VAF and indicates that VAF decreased in all responder patients and in 67% (4/6) of SD patients. FIG. 3B shows that NSCLC patients having a decrease in mean VAF generally have a greater chance of longer progression free survival and overall survival relative to patients exhibiting an increase in mean VAF.

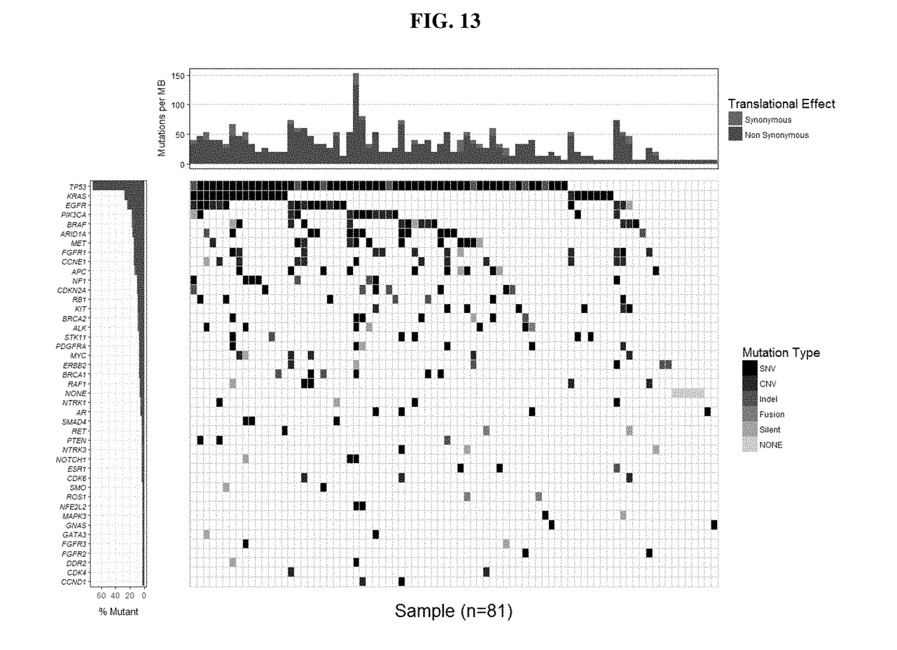

Analysis of patient samples in a second NSCLC clinical trial (ATLANTIC) evaluating anti-PD-L1 antibody therapy (durvalumab) confirms that ctDNA is detectable in cell-free DNA isolated from plasma in a high percentage of patients. Analysis of samples obtained from 88 patients during screening identified variants in 94% of the samples (83/88), with several previously observed variants and new frequently observed variants, including TP53 (72% of samples), PIKCA (16% of samples), EGFR (22% of samples), KRAS (27% of samples), and ARID1A (16% of samples). See FIG. 13.

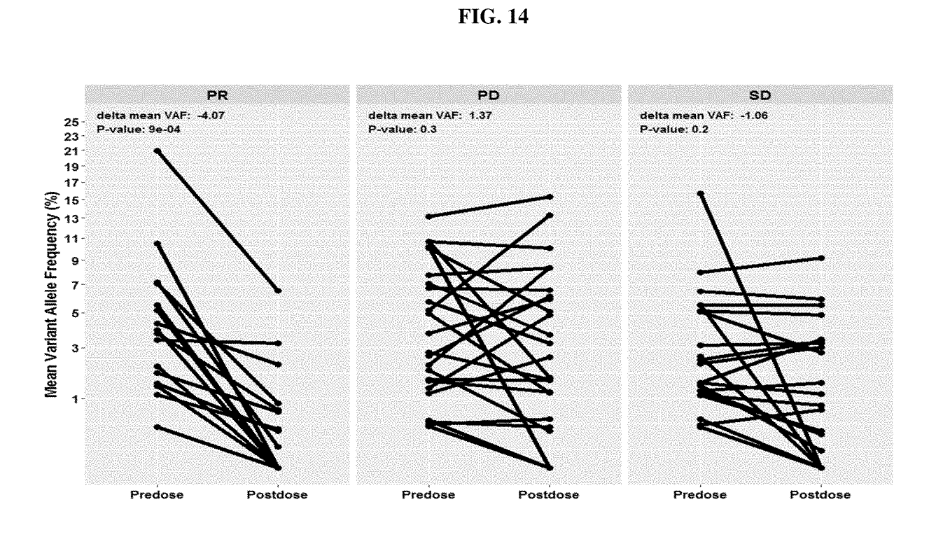

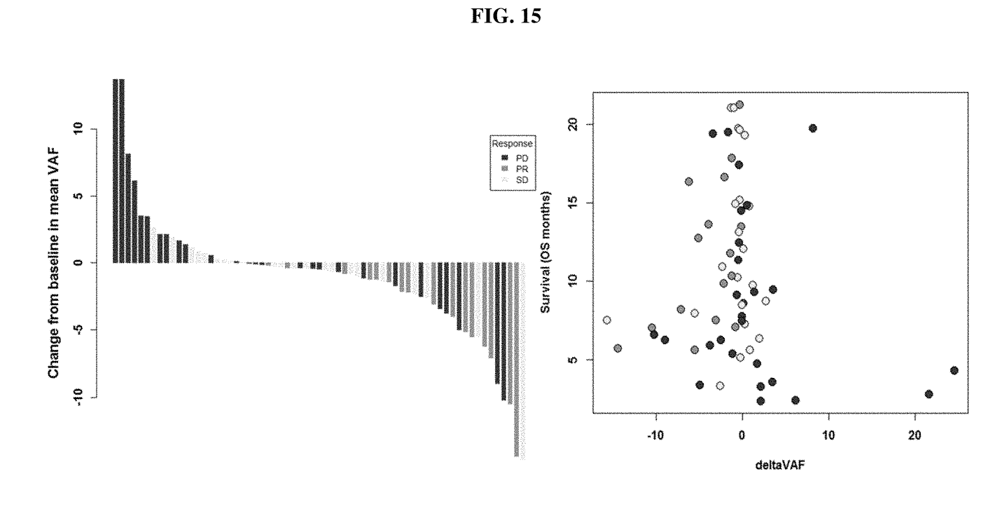

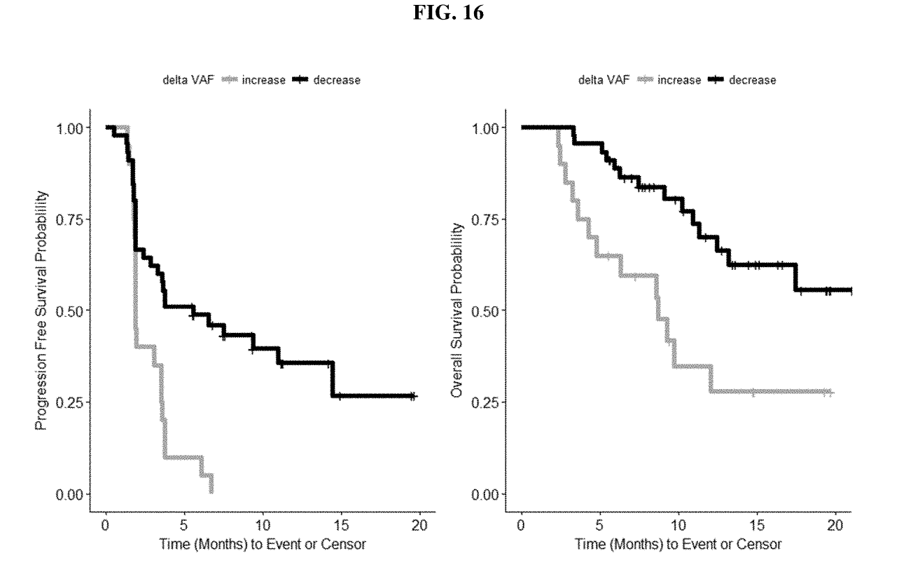

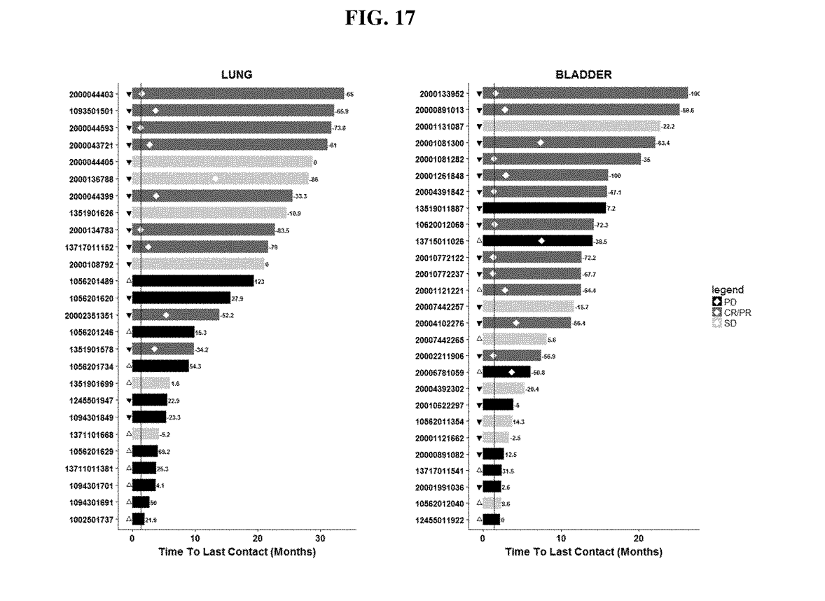

As observed in both NSLC studies, changes in mean variant allele frequencies (VAF) during the course of therapy correlates with disease progression. Responding patients (PR) have a significant decrease (−4.07%, p=0.0009) in ctDNA VAF after 4 doses/treatments, patients with stable disease (SD) have a smaller decrease in ctDNA VAF (−1.06, p=0.2) while non-responders have an observable increase in mean VAF (+1.37%, p=0.3). See FIG. 14. In FIG. 15 patient response is plotted as a function of the change in mean VAF and indicates that VAF decreased in all responder patients and in 64% (14/22) of SD patients. FIG. 16 depicts the PFS probability and OS probability for patients exhibiting a mean decrease or increase in VAF after 6 weeks of durvalumab dosing. As is shown from study CP1108, these data indicate that an observed reduction in mean VAF as early as 6 weeks after initiation of therapy may be predictive of longer PFS and OS with treatment comprising durvalumab. Individual patient response for lung and bladder cancer patients is presented in FIG. 17.

### Example 2

### Decrease in ctDNA Mutation Burden Observed in Responding NSCLC Patients

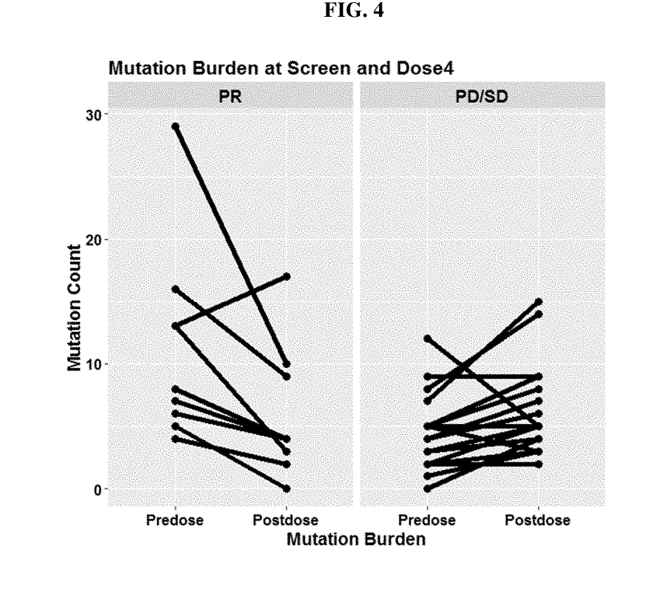

In addition to the decrease in mean VAF discussed in Example 1, responding patients (PR) exhibit a significant decrease in mutation burden as determined by total mutation counts (average difference of −5.3, p=0.037, and ci(95%)=−10.2, −0.4) after 8 weeks of treatment when compared to non-responding (PD/SD) patients (average difference of +1.6, p=0.036, ci(95%)=0.1, 3.1). See FIG. 4 which plots the total mutation count at screening (predose) and after dose 4 administered at week 6 (postdose).

### Example 3

### New ctDNA Mutations are Associated in Patients with Progressive NSCLC

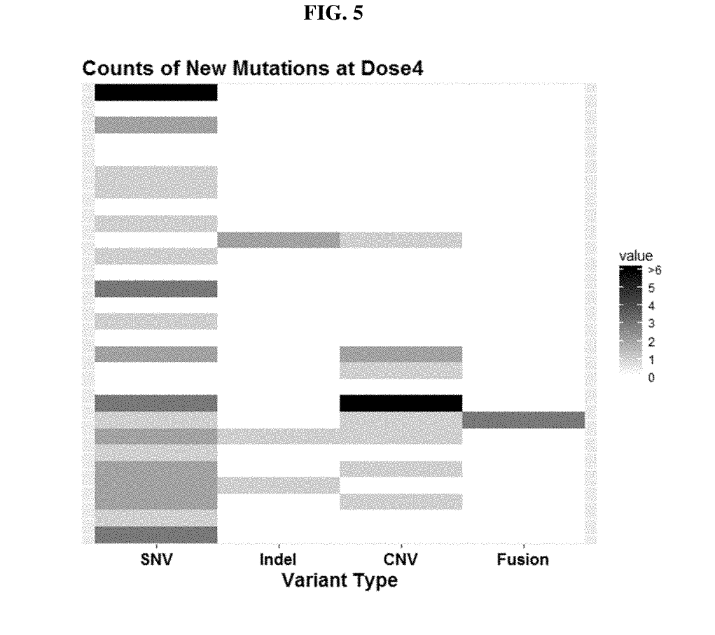

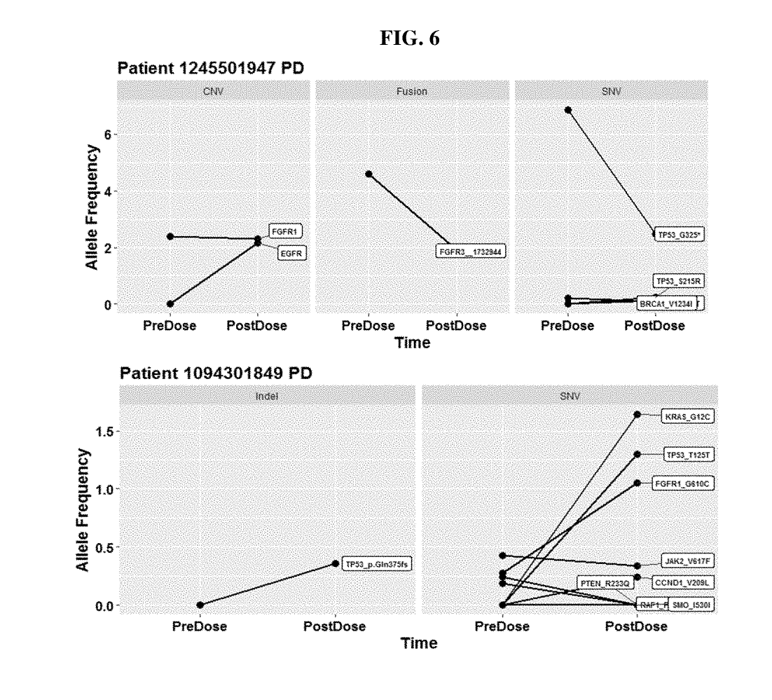

New ctDNA mutations are detected in 100% of PD patients after 8 weeks (4 doses), compared to 57% of patient classified as SD and 56% of patients classified as PR. See FIG. 5. The new mutations detected in PR patients (5/9) do not include driver mutations common to NSCLC, while driver mutations common to NSCLC were detected in 42% of PD patients (5/12) and 14% of SD patients (1/7). As such, during the course of therapy detection of mutations absent at screen are more likely to be associated with non-responder (PD) patients. FIG. 6 provides examples of new driver mutations appearing in two non-responder patients (EGFR, TP53(S215R), and ALK(M1302T); and TP53(p.Gln375fs), KRAS(G12C), and TP53(T125T)).

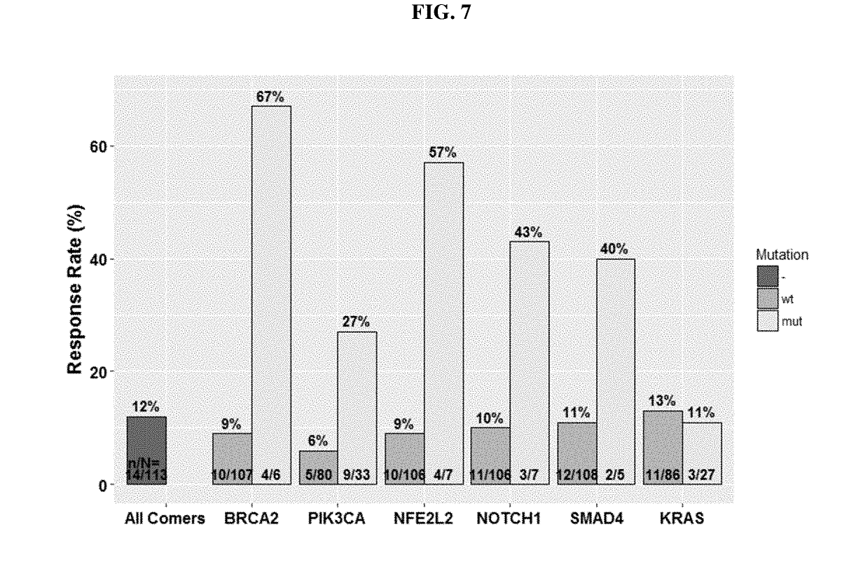

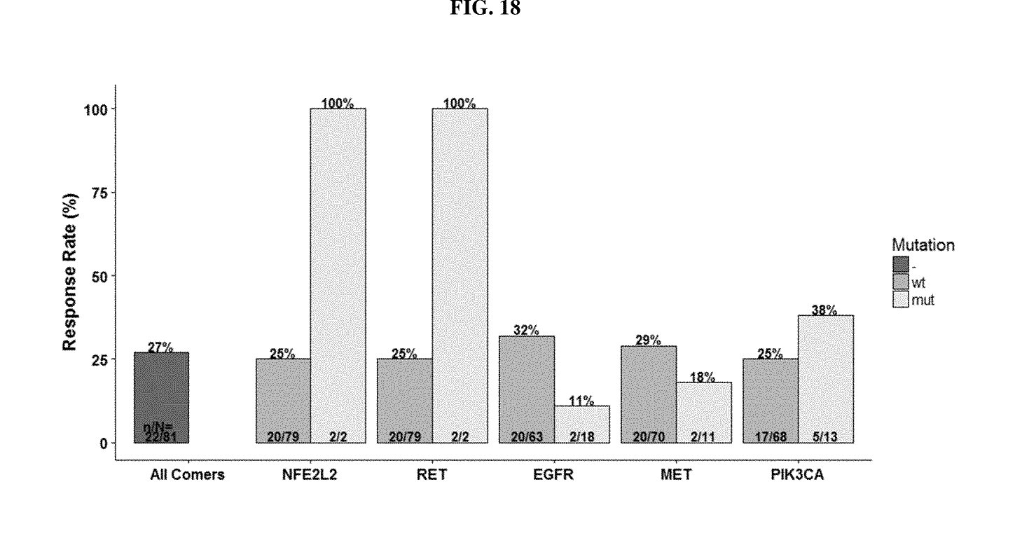

Further analysis of the data identified that several ctDNA variants which can be detected at baseline (screen) are associated with responders, including BRCA1, BRCA2, PIK3CA, NFE2L2, NOTCH1, SMAD4, and KRAS and may be useful in providing early patient prognosis and stratification as a good candidate for anti-PD-L1 antibody therapy (e.g., durvalumab). Table 1a identifies the variants show an increase in ORR, that BRCA2 and NFE2L2 may be associated with improvements in median PFS, and that all mutations except PIK3CA may be associated with improvement in OS. See also, FIG. 7. BRCA2 (OR, 18.4; Pval 0.002); PIK3CA (OR, 5.5; Pval 0.004); NFE2L2 (OR, 12.2; Pval 0.004); NOTCH1 (OR, 6.3; Pval 0.040); SMAD4 (OR, 5.2; Pval 0.115; KRAS (OR, 0.9; Pval 1.000); FIG. 18. NFE2L2 (100% response, Pval 0.071); RET (100% response, Pval 0.071); EGFR (0.3 response, Pval 0.135); MET (0.27 response, Pval 0.272); PIK3CA (1.86 response, Pval 0.325). BRCA1/2, NFE2L2, NOTCH1, and PIK3CA mutations in particular may be more commonly identified among responders prior to anti-PD-L1 antibody therapeutic intervention.

TABLE 1a
ctDNA mutations associated with potential response to durvalumab

| | ctDNA mutation | | | | |
|---|---|---|---|---|---|
| | BRCA2 | NFE2L2 | PIK3CA | NOTCH1 | SMAD4 |
| Endpoint | (6/110)* | (7/109)* | (34/82)* | (7/109)* | (5/111)* |
| ORR* % | 67/9 | 57/9 | 27/6 | 43/10 | 40/11 |
| Median PFS, | 7.2/1.4 | 7.4/1.4 | 1.4/1.4 | 1.4/1.4 | 1.7/1.4 |
| months* | P = 0.01 | P = 0.02 | P = 0.17 | P = 0.23 | P = 0.24 |
| Median OS, | NR/9.1 | 25.6/9.4 | 9.4/9.8 | 25.6/9.1 | 22.7/9.1 |
| months* | P = 0.05 | P = 0.44 | P = 0.63 | P = 0.13 | P = 0.35 |

NR: not reached
*N and effect size with/without mutation
NE omitted from ORR calculation

TABLE 1b
ctDNA mutations associated with
potential negative/positive ORR to durvalumab

| Gene | OR | Pval |
|---|---|---|
| NFE2L2 | INF | 0.071 |
| RET | INF | 0.071 |
| EGFR | 0.30 | 0.135 |
| RB1 | 0 | 0.181 |
| SMO | INF | 0.272 |
| CCND2 | INF | 0.272 |
| GATA3 | INF | 0.272 |
| DDR2 | INF | 0.272 |
| MET | 0.27 | 0.272 |
| PIK3CA | 1.86 | 0.325 |
| ALK | 2.90 | 0.337 |
| BRCA2 | 2.90 | 0.337 |
| BRCA1 | 2.90 | 0.337 |
| CDKN2A | 2.15 | 0.382 |
| TP53 | 1.73 | 0.423 |

### Example 4

### Decrease in ctDNA Mean Variant Allele Frequency Observed in Responding Bladder Cancer Patients

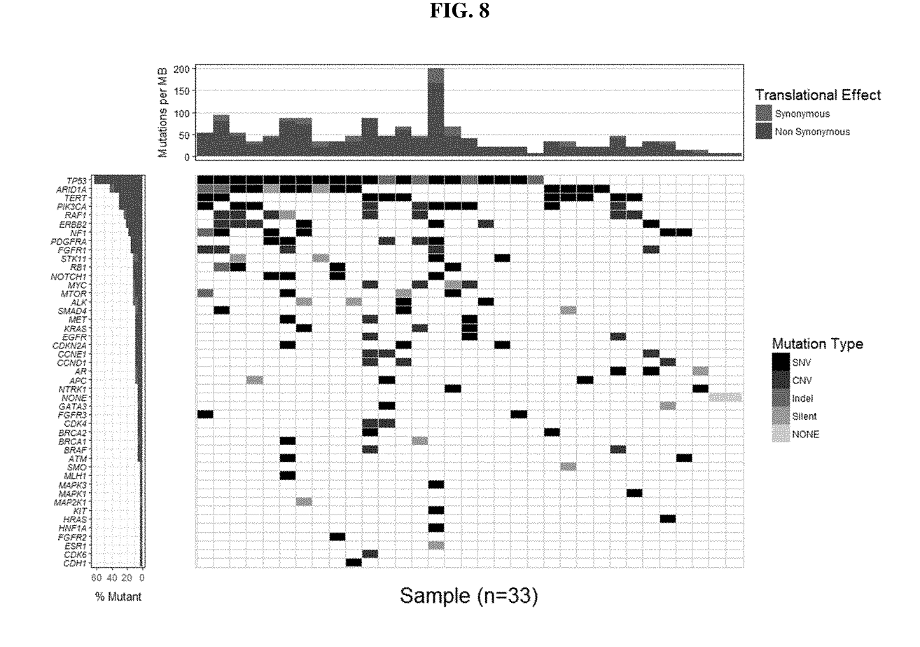

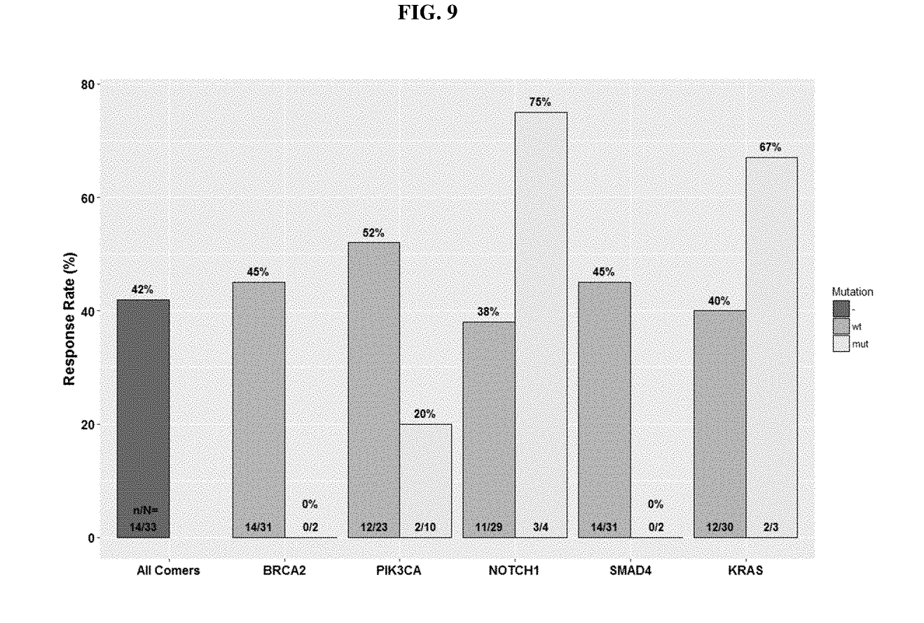

Analysis of samples obtained from 33 patients during screening identified variants in 94% of the samples (31/33), with several frequently observed variants, including TP53 (73% of samples), ARID1A (55% of samples), PIK3CA (39% of samples), ERBB2 (33% of samples), and TERT (33% of samples), and CDKN2A (16% of samples). See FIG. 8. Several genes having mutations that correlated with NSCLC response were also identified in bladder cancer patients, with NOTCH1 likely being associated with an increased response rate. (FIG. 9).

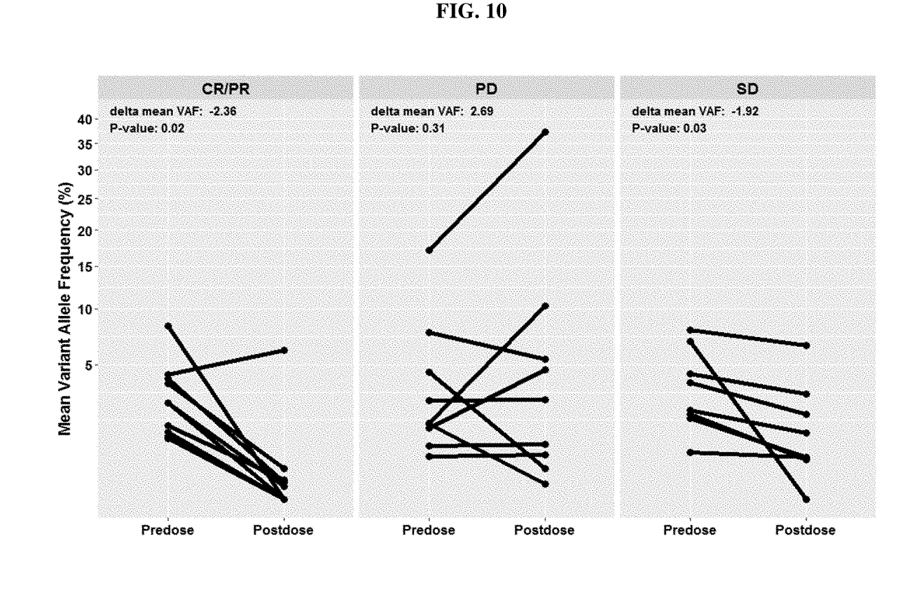

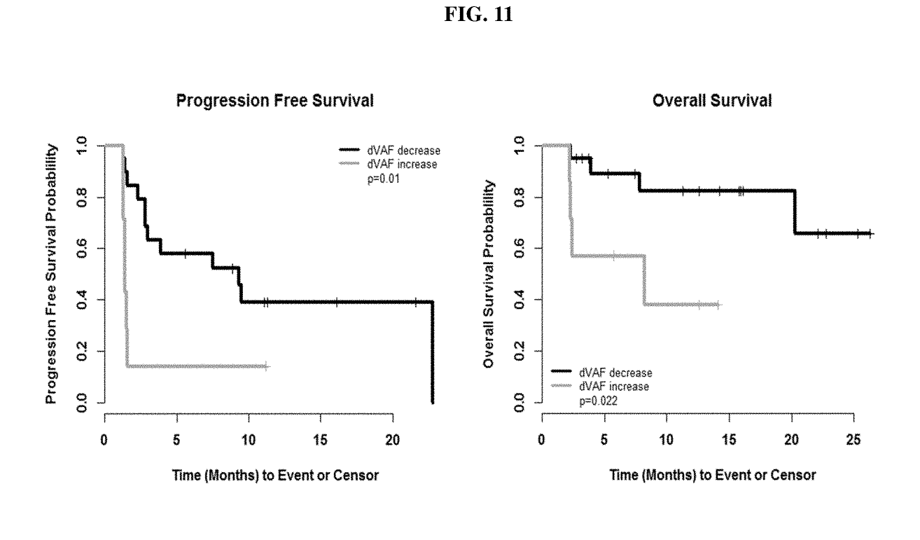

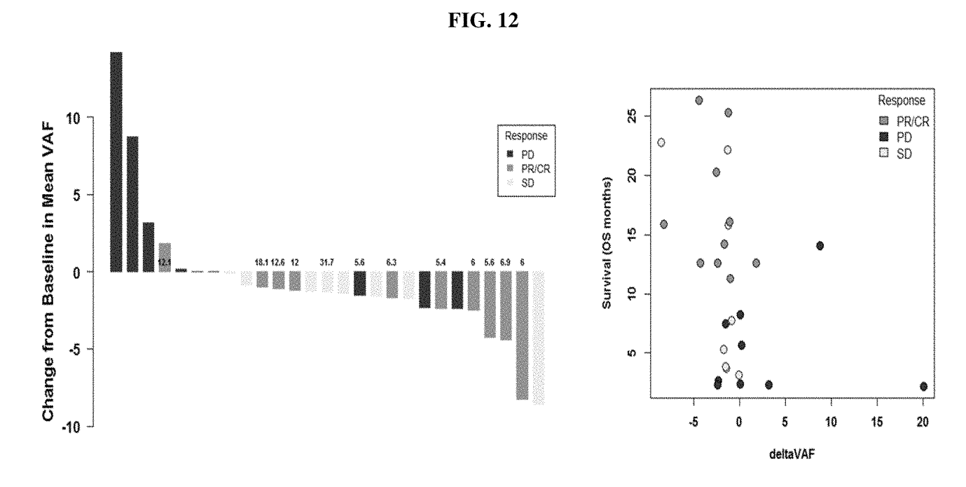

As observed in the data associated with response in NSCLC patients, changes in mean VAF over the course of therapy correlates with bladder cancer disease progression. Responding patients (CR/PR) have a significant decrease (−2.36%, p=0.02) in ctDNA VAF after 4 doses/treatments while non-responders (PD) have an observable increase in mean VAF (+2.69%, p=0.31). See FIG. 10. In particular, improved PFS and OS probabilities were strongly associated with a decrease in mean VAF following durvalumab dosing, as shown in FIG. 11. FIG. 12 plots patient response as a function of the change in mean VAF and indicates that VAF decreased in all responder (PR/CR) patients except for one and in all SD patients.

Pooling Data to Identify Screen Mutations Most Frequently Associated with Response

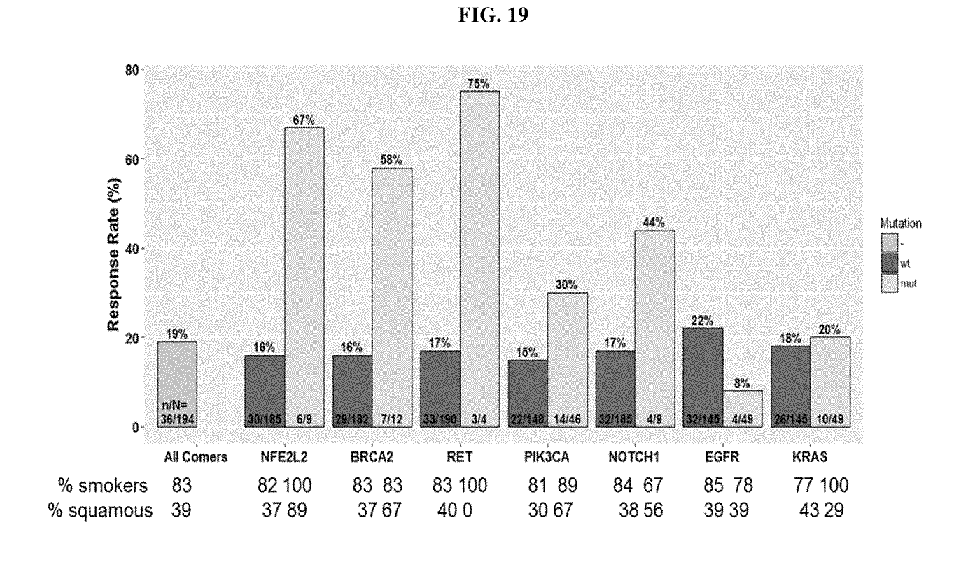

Pooling the mutation data from the clinical trials discussed allowed for identified of the top 15 mutations detected in predose sampling that were associated with response for squamous and non-squamous carcinomas. Table 2a summarizes the genes and Table 2b identifies mutations likely having a benefit, as well as resistance mutations (EGFR or STK11 in responders and non-responders. FIG. 19 provides a histogram for a selected number of the identified variants, identifying the % of patients classified as smokers and having squamous cell carcinomas (194 total patients).

TABLE 2a
Top 15 gene mutation associations ranked by p-value

| Gene | OR | Pval |
|---|---|---|
| NFE2L2 | 10.142 | 0.002 |
| BRCA2 | 7.272 | 0.002 |
| RET | 13.986 | 0.021 |
| PIK3CA | 2.492 | 0.028 |
| EGFR | 0.315 | 0.033 |
| NOTCH1 | 3.789 | 0.063 |
| BRCA1 | 3.002 | 0.069 |
| ALK | 2.679 | 0.125 |
| CCNE1 | 2.132 | 0.150 |
| HNF1A | INF | 0.186 |
| GATA3 | INF | 0.186 |
| DDR2 | INF | 0.186 |
| STK11 | 0.000 | 0.214 |
| TP53 | 1.713 | 0.237 |
| TERT | 4.437 | 0.337 |

TABLE 2b
Mutations associated with benefit, and percent of resistance mutations in
responder/non-responder patients.

| Mutations | Resistance Mutations (EGFR or STK11) | | | |
|---|---|---|---|---|
| with Benefit | Responders | | Non-Responders | |
| BRCA1 | 2/5 | (40% | 1/8 | (13%) |
| BRCA2 | 0/7 | (0%) | 3/5 | (60%) |
| NFE2L2 | 1/6 | (17% | 1/3 | (33%) |
| NOTCH1 | 1/4 | (25%) | 2/5 | (40%) |
| PIK3CA | 1/14 | (7%) | 17/32 | (53%) |

The above Examples illustrate that methods comprising detection of ctDNA can identify particular ctDNA mutations that are associated with diagnosis, prognosis, and treatment of cancers that may be responsive to an anti-PD-L1 antibody therapy. As such, the methods provide for identification and stratification patients who may benefit from anti-PD-L1 antibody therapy. Several mutations in ctDNA markers, including NOTCH1, BRCA2, BRCA2, PIK3CA, and NFE2L2, can be identified upon initial screening of subjects suffering from lung cancer or bladder, and identify them as likely responders to treatment with an anti-PD-L1 antibody.

Furthermore, the data demonstrates that anti-PD-L1 antibody therapy is associated with a higher likelihood of positive outcomes (increased OS and PFS) when the therapy induced a decrease in total ctDNA mutation burden or a decrease in mean VAF during or upon completion of therapy.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

SEQUENCE LISTING

SEQ ID NO: 1
EIVLTQSPOTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
SEQ ID NO: 2
EVQLVESOGGLVQPGGSLRLSCAASOFTFSRYWMSWVRQAPGKOLEWVAN
IKQDGSEKYYVDSVKGRFTISRDNAKNSLYLQMNSLRAEDTAVYYCAREG
GWFGELAFDYWGQGTLVTVSS
SEQ ID NO: 3-VH CDR1
GFTFSRYWMS
SEQ ID NO: 4-VH CDR2
NIKQDGSEKYYVDSVKG
SEQ ID NO: 5-VH CDR3
EGGWFGELAFDY
SEQ ID NO: 6-VL CDR1
RASQRVSSSYLA
SEQ ID NO: 7-VL CDR2
DASSRAT
SEQ ID NO: 8-VL CDR3
QQYGSLPWT

1. A method of treatment comprising administering an anti-PD-L1 antibody, or an antigen binding fragment thereof, to a patient identified as having a cancer that expresses a mutation in one or more circulating tumor DNA (ctDNA) markers comprising BRCA1, BRCA2, PIK3CA, NFE2L2, NOTCH1, SMAD4, ARID1A, APC, or KRAS.

2. The method of claim 1, wherein the cancer is selected from lung cancer, bladder cancer, and head and neck cancer.

3. The method of claim 2, wherein the cancer is lung cancer and the mutation in one or more ctDNA markers comprise BRCA1, BRCA2, NFE2L2, PIK3CA, or NOTCH1.

4. The method of claim 2, wherein the cancer is lung cancer and the mutation in one or more ctDNA markers comprise BRCA2 and NFE2L2.

5. The method of claim 3, wherein the lung cancer is non-small cell lung cancer (NSCLC).

6. The method of claim 2, wherein the cancer is bladder cancer and the mutation in one or more ctDNA markers comprises BRCA1, BRCA2, ARID1A, APC, PIK3CA, or NOTCH1.

7. The method of claim 1, wherein the anti-PD-L1 antibody is durvalumab.

8. A method of treatment comprising administering durvalumab or an antigen binding fragment thereof to a patient identified as having a cancer that expresses a mutation in one or more ctDNA markers comprising BRCA1, BRCA2, PIK3CA, NFE2L2, NOTCH1, SMAD4, ARID1A, APC, or KRAS.

9. The method of claim 1, wherein the patient is identified as responsive to durvalumab.

10. The method of any claim 1, wherein the patient is further identified as having a tumor expressing PD-L1.

11. The method of claim 5, wherein the NSCLC is selected from the group consisting of squamous cell carcinoma, non-squamous cell carcinoma, adenocarcinoma, large cell carcinoma, adenosquamous carcinoma and sarcomatoid carcinoma.

12. The method of claim 1, wherein at least about 0.1, about 0.3, about 1, about 3, about 10, or about 15 mg/kg durvalumab, or an antigen-binding fragment thereof, is administered.

13. The method of claim 12, wherein about 1 mg/kg durvalumab, or an antigen-binding fragment thereof, is administered.

14. The method of claim 12, wherein about 3 mg/kg durvalumab, or an antigen-binding fragment thereof, is administered.

15. The method of claim 12, wherein about 10 mg/kg durvalumab or an antigen-binding fragment thereof is administered.

16. The method of claim 12, wherein about 15 mg/kg durvalumab, or an antigen-binding fragment, thereof is administered.

17. The method of claim 1, wherein the administration is repeated about every 14 or 21 days.

18. The method of claim 1, wherein at least two doses are administered.

19. The method of claim 1, wherein at least three doses are administered.

20. The method of claim 1, wherein at least four doses are administered.

21. A method for characterizing the responsiveness of a cancer in a patient to an anti-PD-L1 antibody treatment, the method comprising:
detecting variant allele frequency in ctDNA in a first plasma sample taken from the patient at a first time point,
detecting variant allele frequency in ctDNA in at least a second plasma sample taken from the patient at least at a second time point, and
determining the difference of the variant allele frequency in ctDNA between the first and at least second plasma samples,
wherein a decrease in the variant allele frequency in the at least second plasma sample relative to the first plasma sample characterizes the cancer as responsive to anti-PD-L1 antibody treatment.

22. The method according to claim 21, further comprising administering the anti-PD-L1 antibody to the patient after the first plasma sample is taken from the patient.

23. The method according to claim 21, wherein the variant allele frequency in ctDNA is determined by total mutation count in the first sample and in the at least second sample.

24. The method according to claim 21, wherein the variant allele frequency in ctDNA is determined by the mean variant allele frequency in the first sample and in the at least second sample.

25. The method of claim 24, further comprising detecting PD-L1 expression in the tumor.

26. A method of determining the efficacy of anti-PD-L1 therapeutic antibody treatment in a patient having lung cancer or bladder cancer comprising
detecting variant allele frequency in ctDNA in a first plasma sample taken from the patient at a first time point,
administering anti-PD-L1 therapeutic antibody to the patient after obtaining the first plasma sample,
detecting variant allele frequency in ctDNA in at least a second plasma sample taken from the patient at least at a second time point after administration of the anti-PD-L1 therapeutic antibody, and
determining the difference of the variant allele frequency in ctDNA between the first and at least second plasma samples,
wherein a decrease in the variant allele frequency in the at least second plasma sample relative to the first plasma sample identifies the anti-PD-L1 antibody treatment as effective.

27. The method according to claim 26, wherein the variant allele frequency in ctDNA is determined by total mutation count in the first sample and in the at least second sample.

28. The method according to claim 26, wherein the variant allele frequency in ctDNA is determined by the mean variant allele frequency in the first sample and in the at least second sample.

29. A method of identifying a patient having a cancer responsive to an anti-PD-L1 antibody, the method comprising detecting the expression of a mutation in one or more circulating tumor DNA (ctDNA) markers comprising BRCA1, BRCA2, PIK3CA, NFE2L2, NOTCH1, SMAD4, ARID1A, APC, or KRAS.

30. The method of claim 29, wherein the cancer is selected from lung cancer, bladder cancer, and head and neck cancer.

31. The method of claim 30, wherein the cancer is lung cancer and the mutation in one or more ctDNA markers comprise BRCA1, BRCA2, NFE2L2, PIK3CA, or NOTCH1.

32. The method of claim 30, wherein the cancer is lung cancer and the mutation in one or more ctDNA markers comprise BRCA2 and NFE2L2.

33. The method of claim 31, wherein the lung cancer is non-small cell lung cancer (NSCLC).

34. The method of claim 30, wherein the cancer is bladder cancer and the mutation in one or more ctDNA markers comprises BRCA1, BRCA2, ARID1A, APC, PIK3CA, or NOTCH1.

35. The method of claim 29, wherein the anti-PD-L1 antibody is durvalumab.

36. The method of claim 33, wherein the NSCLC is selected from the group consisting of squamous cell carcinoma, non-squamous cell carcinoma, adenocarcinoma, large cell carcinoma, adenosquamous carcinoma and sarcomatoid carcinoma.

37. A method of treating a patient identified as having cancer, the method comprising:
detecting variant allele frequency in one or more ctDNA markers in a first plasma sample taken from the patient at a first time point,
administering an anti-PD-L1 therapeutic antibody to the patient after obtaining the first plasma sample,
detecting variant allele frequency in one or more ctDNA markers in at least a second plasma sample taken from the patient at least at a second time point after administration of the anti-PD-L1 therapeutic antibody, and
determining the difference of the variant allele frequency in one or more ctDNA markers between the first and at least second plasma samples,
wherein a decrease in the variant allele frequency in the at least second plasma sample relative to the first plasma sample identifies the anti-PD-L1 antibody treatment as effective, and wherein the one or more circulating tumor DNA (ctDNA) markers comprise BRCA1, BRCA2, PIK3CA, NFE2L2, NOTCH1, SMAD4, ARID1A, APC, or KRAS.

38. The method of claim 37, wherein the cancer is selected from lung cancer, bladder cancer, and head and neck cancer.

39. The method of claim 38, wherein the cancer is lung cancer and the mutation in one or more ctDNA markers comprise BRCA1, BRCA2, NFE2L2, PIK3CA, or NOTCH1.

40. The method of claim 39, wherein the cancer is lung cancer and the mutation in one or more ctDNA markers comprise BRCA2 and NFE2L2.

41. The method of claim 39, wherein the lung cancer is non-small cell lung cancer (NSCLC).

42. The method of claim 38, wherein the cancer is bladder cancer and the mutation in one or more ctDNA markers comprises BRCA1, BRCA2, ARID1A, APC, PIK3CA, or NOTCH1.

43. The method of claim 37, wherein the anti-PD-L1 antibody is durvalumab.

44. The method of claim 37, wherein the patient is further identified as having a tumor expressing PD-L1.

45. The method of claim 41, wherein the NSCLC is selected from the group consisting of squamous cell carcinoma, non-squamous cell carcinoma, adenocarcinoma, large cell carcinoma, adenosquamous carcinoma and sarcomatoid carcinoma.

46. The method of claim 37, wherein at least about 0.1, about 0.3, about 1, about 3, about 10, or about 15 mg/kg durvalumab, or an antigen-binding fragment thereof, is administered

47. The method of claim 46, wherein about 1 mg/kg durvalumab, or an antigen-binding fragment thereof, is administered.

48. The method of claim 46, wherein about 3 mg/kg durvalumab, or an antigen-binding fragment thereof, is administered.

49. The method of claim 46, wherein about 10 mg/kg durvalumab or an antigen-binding fragment thereof is administered.

50. The method of claim 46, wherein about 15 mg/kg durvalumab, or an antigen-binding fragment, thereof is administered.

51. The method of claim 37, wherein the administration is repeated about every 14 or 21 days.

52. The method of claim 37 wherein at least two doses are administered.

53. The method of claim 37, wherein at least three doses are administered.

54. The method of claim 37, wherein at least four doses are administered.

55. A method of treating a patient having a cancer comprising:
identifying whether the patient will be responsive to an anti-PD-L1 antibody by detecting the expression of a mutation in one or more circulating tumor DNA (ctDNA) markers comprising BRCA1, BRCA2, PIK3CA, NFE2L2, NOTCH1, SMAD4, ARID1A, APC, or KRAS; and
treating the patient with a therapy other than an anti-PD-L1 antibody if the one or more ctDNA markers is not expressed.


SEQUENCE LISTING (10 sequences)

SEQ ID NO: 1 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Artificial Sequence
  Description of Artificial Sequence Synthetic peptide
GFTFSRYWMS

SEQ ID NO: 4 - protein, 17 aa, Artificial Sequence
  Description of Artificial Sequence Synthetic peptide
NIKQDGSEKYYVDSVKG

SEQ ID NO: 5 - protein, 12 aa, Artificial Sequence
  Description of Artificial Sequence Synthetic peptide
EGGWFGELAFDY

SEQ ID NO: 6 - protein, 12 aa, Artificial Sequence
  Description of Artificial Sequence Synthetic peptide
RASQRVSSSYLA

SEQ ID NO: 7 - protein, 7 aa, Artificial Sequence
  Description of Artificial Sequence Synthetic peptide
DASSRAT

SEQ ID NO: 8 - protein, 9 aa, Artificial Sequence
  Description of Artificial Sequence Synthetic peptide
QQYGSLPWT

SEQ ID NO: 9 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa